(19) United States
(12) Patent Application Publication
(10) Pub. No.: US 2016/0271418 A1
(43) Pub. Date: Sep. 22, 2016

(54) THERAPEUTIC LIGHT ENABLED TOILET AND METHODS FOR OPERATING A THERAPEUTIC LIGHT SOURCE

(71) Applicant: AKARI SYSTEMS, INC., Sunnyvale, CA (US)

(72) Inventor: Philip Ferolito, Sunnyvale, CA (US)

(21) Appl. No.: 15/072,302

(22) Filed: Mar. 16, 2016

Related U.S. Application Data
(60) Provisional application No. 62/177,410, filed on Mar. 16, 2015.

(51) Int. Cl. A61N 5/06

(52) U.S. Cl. CPC A61N 5/0613; A61N 2005/0626; A61N 2005/0643; A61N 2005/0661

(57) ABSTRACT

A device for therapeutic irradiation with light of a portion of a person's skin located on the posterior between the lumbus and the popliteal fossa during the use of a toilet, may comprise: a light source; a controller electrically coupled to the light source, the controller being configured to control the intensity and duration of light emitted from the light source during a therapeutic session; one or more sensors comprising a first sensor coupled to the controller for detecting a person seated on the toilet; and a power source electrically coupled to the light source and the controller; wherein the controller is further configured to turn on, and keep turned on for the duration of the therapeutic session, the light source when the first sensor detects the person seated on the toilet, and wherein the light source is configured to illuminate the portion of the person's skin.

### CROSS-REFERENCE TO RELATED APPLICATIONS

This application claims the benefit of U.S. Provisional Application No. 62/177,410 filed Mar. 16, 2015, incorporated by reference in its entirety herein.

### FIELD OF THE INVENTION

The present invention relates generally to a light source with therapeutic benefits administered while using a toilet, and more specifically, although not exclusively, to an ultraviolet, blue, red, near infrared, and/or infrared light source utilizing light to create, activate and or mobilize molecules affecting regions of the body not exposed to the light. These effects include but are not limited to the promotion of wellbeing, management of pain, healing of wounds, stimulation of vitamin D synthesis, reduction of inflammation, regulation of immune response, reduction of blood pressure, and abatement of seasonal depression.

### BACKGROUND

Adoption of new treatments, especially those associated with long term prevention of disease benefit greatly when they may be administered through an existing activity requiring no behavioral changes to the general population. A fine example of this is the introduction of Iodine in table salt in the United States early in the 20^{th} century, effectively eliminating much of the existing thyroid disease without requiring individuals to alter their normal behavior even though the alternative was simply to take a supplement.

Many research publications (over 3000 of them in 2013) show beneficial association with low intensity ultraviolet radiation (UVR) skin exposure and a host of chronic health conditions. Unfortunately not everyone lives in a geographic location where sunlight contains the required ultraviolet wavelengths year round, resulting in a seasonal fluctuation of critical molecules/hormones in a person's blood, such as vitamin D. In addition to geographical factors influencing insufficient sunlight exposure, modem lifestyles and working habits prevent the bulk of the population from spending adequate time in the sun during the hours when the required wavelengths are present. For example in Boston during the summer, the time period when sunlight exposure can produce vitamin D is between 10 am and 3 pm; a range of time where most individuals are working indoors. In winter, even the noon sun is not sufficient to synthesize vitamin D in the skin.

It has been established that human skin will convert naturally occurring 7-dehydrocholesterol in to pre vitamin D3 which will then begin a process by which serum 25(OH)D levels are raised. Required light wavelengths are between 280 mn and 320 run and the most effective rate of conversion has been determined to be around 298 mn. The range from 280 mn to 320 mn is in the UVB (ultraviolet B) part of the spectrum and long-term exposure to the eyes is discouraged as it is responsible for increased risks of cataracts and other ocular damage. Due to the potential for long term eye damage, UV filtering sun glasses are recommended when out in the direct sunlight and it would be considered poor practice to intentionally add the 280 nm through 320 nm wavelength light to indoor lighting. Additionally those skilled in the art know that wavelengths less than 290 nm can do considerable direct and indirect damage to DNA and exposure should be avoided.

In addition to endogenous synthesis of vitamin D, light has been shown to produce hundreds of photo-products in skin. Many of these molecules are highly mobile allowing systemic, whole body, effects despite localized generation. These molecules include but are not limited to the production of cis-urocyanic acid, nitric oxide, beta-endorphine and the hormone vitamin D. Each activates different, though possibly sympathetic (interrelated), pathways bringing about positive health benefits. These benefits include but are not limited to: vitamin D regulating calcium absorption, bone mineralization and the overall maintenance of calcium homeostasis which is responsible for skeletal health as well as positive effects on vitamin D receptors in virtually all other systems in the human body; mobilization of nitrite in the skin to form nitric oxide, a molecule reacting with the smooth muscles lining arteries, reducing blood pressure, a known risk factor for heart disease. Production of beta-endorphine can help manage pain by interacting with the brain in a manner similar to an opiate.

Human populations are moving steadily away from equatorial regions, modem lifestyles keep many persons indoors all day, and social customs of sun avoidance all contribute to large populations of humans who no longer have access to natural sources of light capable of producing these molecules.

There is a need for therapeutic light sources suitable for efficient and convenient treatment of human patients, for enabling ultraviolet, blue, red, near infrared, and/or infrared light therapy for the promotion of wellbeing, management of pain, healing of wounds, stimulation of vitamin D synthesis, reduction of inflammation, regulation of immune response, reduction of blood pressure, etc.

### SUMMARY OF THE INVENTION

According to some embodiments, a device for therapeutic irradiation with light of a portion of a person's skin located on the posterior between the lumbus and the popliteal fossa during the use of a toilet, may comprise: a light source; a controller electrically coupled to the light source, the controller being configured to control the intensity of light emitted from the light source and the duration of emission of light from the light source during a therapeutic session; one or more sensors comprising a first sensor for detecting a person seated on the toilet, the first sensor being electrically coupled to the controller; and a power source electrically coupled to the light source and the controller; wherein the controller is further configured to turn on, and keep turned on for the duration of the therapeutic session, the light source when the first sensor detects the person seated on the toilet, and wherein the light source is configured to illuminate the portion of the person's skin located on the posterior between the lumbus and the popliteal fossa.

According to some embodiments, a method of irradiating with therapeutic light a portion of a person's skin located on the posterior between the lumbus and the popliteal fossa during use of a toilet, may comprise: detecting the presence of a person seated on said toilet using one or more first sensors connected to a controller; on detecting the presence of a person seated on said toilet, enabling by said controller of a light source for illuminating said portion of said person's skin located on the posterior between the lumbus and the popliteal fossa with therapeutic light.

### BRIEF DESCRIPTION OF THE DRAWINGS

FIGS. 1A & 1B show side and front perspective views of an example of a prior art toilet;

FIGS. 2A & 2B show side and bottom views of a prior art toilet seat.

FIGS. 3A & 3B show side and bottom views of an example of a toilet seat configured for light therapy, according to some embodiments of the present invention;

FIG. 4 shows a representation of a human subject seated on a toilet seat, configured as shown in FIGS. 3A & 3B, in a position suitable for light therapy, according to some embodiments of the present invention;

FIG. 5 shows a human anatomical dorsal view, identifying areas of skin that may be subjected to light therapy, according to some embodiments of the present invention;

FIG. 6 shows a schematic representation of a control system for a therapeutic light enabled toilet, according to some embodiments of the present invention;

FIG. 7 shows sensors/light emitters attached to a toilet bowl, according to some embodiments of the present invention;

FIG. 8A & 8B show an insert with sensors/light emitters being placed in position, and in position on a toilet seat, according to some embodiments of the present invention; and

FIG. 9 shows a toilet seat with integrated capacitive sensors, according to some embodiments of the present invention.

### DETAILED DESCRIPTION

Embodiments of the present invention will now be described in detail with reference to the drawings, which are provided as illustrative examples of the invention so as to enable those skilled in the art to practice the invention. Notably, the figures and examples below are not meant to limit the scope of the present invention to a single embodiment, but other embodiments are possible by way of interchange of some or all of the described or illustrated elements. Moreover, where certain elements of the present invention can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

Toilets have undergone steady transitions from a simple hole in the ground at the beginning of the 20^{th} century to the electronically controlled smart toilets now pervasive in countries such as Japan. These personal newer generation toilets are excellent delivery vehicles for therapeutic light as they allow regular treatments, avoid exposure to face and hands, and facilitate exposure of areas of the body least photo-damaged by sunlight.

According to some embodiments, the present invention relates generally to a toilet or toilet components allowing the exposure of a person's skin located on the posterior between the lumbus and the popliteal fossa to specific wavelengths of light while seated on the toilet. Furthermore, one or more sensors detect the presence of a seated individual, and in response the therapeutic light source(s) are enabled, and when the individual is no longer seated, the light source(s) are disabled. Furthermore, one or more timers may be used to assure that exposure from individual wavelengths or groups of wavelengths are limited to generally accepted safe levels. Furthermore, a sensor may be used to estimate the approximate weight of an individual person—this information can be used to help distinguish multiple individuals within a household, allowing the toilet to limit the daily exposure for each individual regardless of the number of seated sessions. Furthermore, a sensor may be used to detect the pigmentation of the seated individual's skin—this information can be utilized to adjust the duration and intensity of the light exposure. (Darker skinned individuals can tolerate, and require, a higher dose of light.) Furthermore, the sensors may be located on the toilet itself, and in embodiments may be located on the toilet seat. Furthermore, the light emitters may be located on the toilet seat and directionally aimed toward the center of the aperture in the toilet seat. The therapeutic light may in embodiments be UV light, light with wavelengths in the range of 290 nm to 400 nm and in embodiments light of wavelength ˜298 mn.

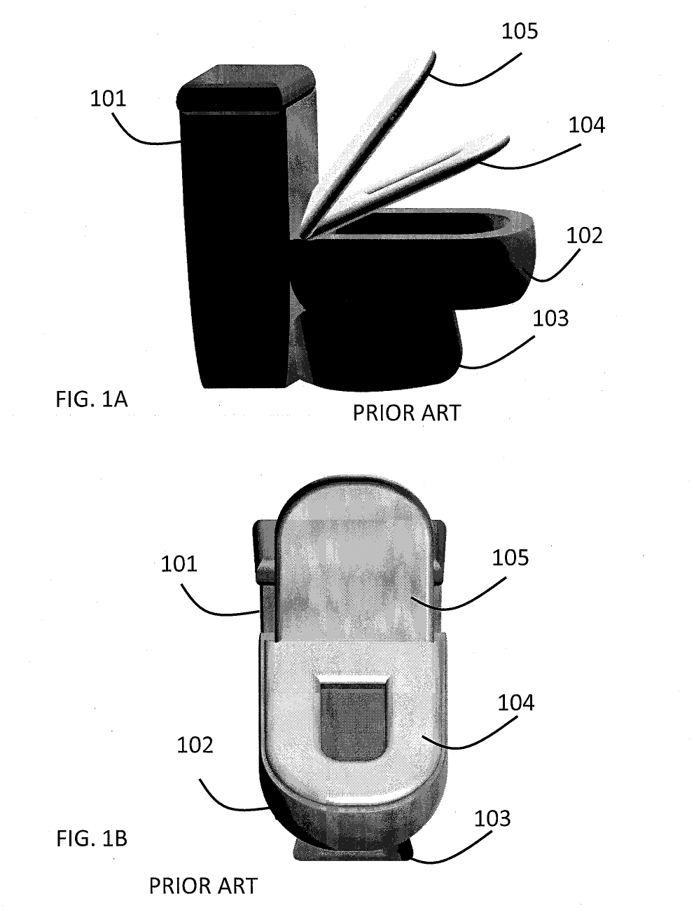

FIGS. 1A & 1B show side and front perspective views of an example of a prior art toilet. A standard toilet can vary in shape and size but is usually composed of a tank 101, a bowl 102, a base 103, a seat 104 and a lid or cover 105. The tank and bowl may be separate and attached units, a unified single unit, or sometimes what is known as a tankless unit. The seat and lid are generally attached at their edges at the back of the bowl, close to the tank; attachment of the seat and cover is usually by a pair of hinges allowing the seat and cover to be raised or lowered independently.

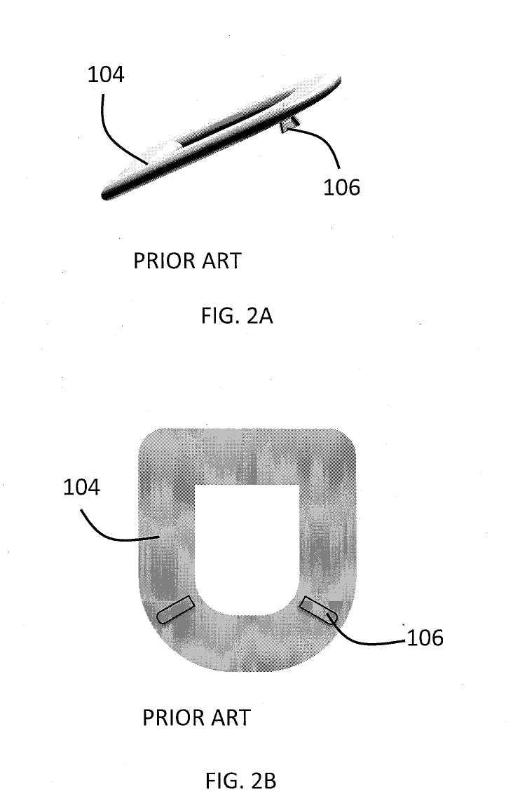

FIGS. 2A & 2B show side and bottom views of a prior art toilet seat 104 with standoffs 106 that support and level the seat on the top lip of the bowl when the seat is in the down position, as when a person is seated upon it.

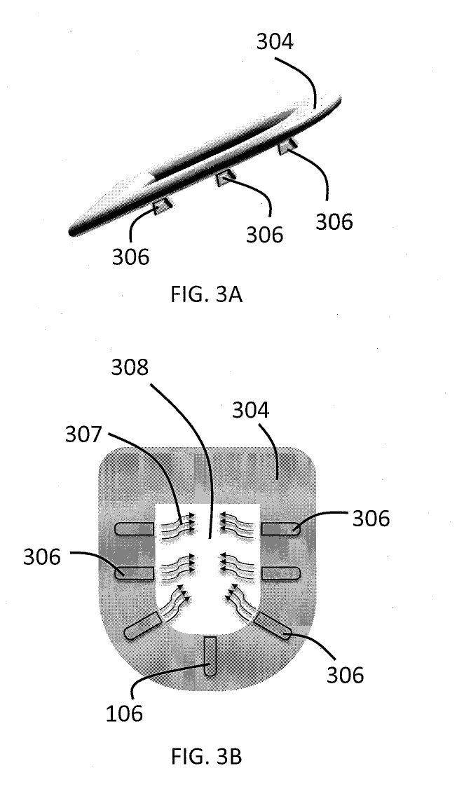

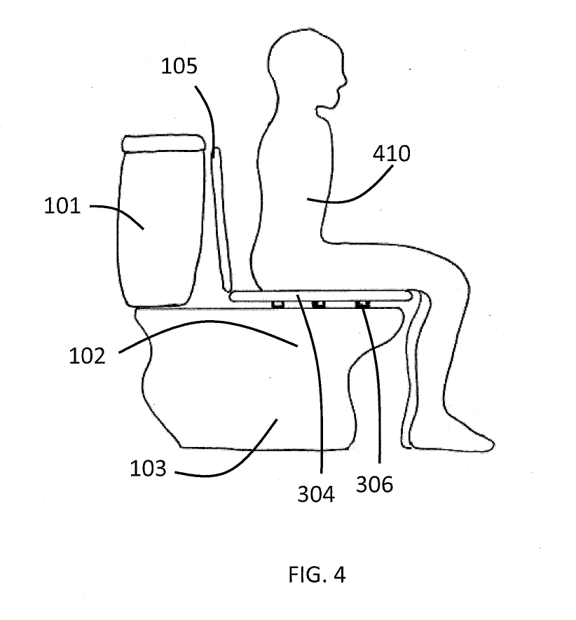

FIGS. 3A & 3B show side and bottom views of an example of a toilet seat configured for light therapy, according to some embodiments of the present invention. In this embodiment the toilet seat 304 has a plurality of standoffs—ordinary standoffs 106 and light emitting standoffs 306—which are configured to emit therapeutic light 307 toward areas of skin of a human subject exposed within the aperture 308 of the toilet seat when the human subject is seated on the toilet as shown in FIG. 4. Furthermore, in embodiments a weight/pressure sensor may be incorporated into one or more of the standoffs 106 and/or 306 for use in the detection of the presence of a seated individual. Note that the precise number and position of the light emitting standoffs is not limited to the specific configurations shown in the figures, but may be varied and yet still provide a useful therapeutic irradiation of the skin of a human subject as described herein. Furthermore, the precise number and position of sensors is not limited to the specific configurations shown in the figures, but may be varied as needed to provide useful data to the controller.

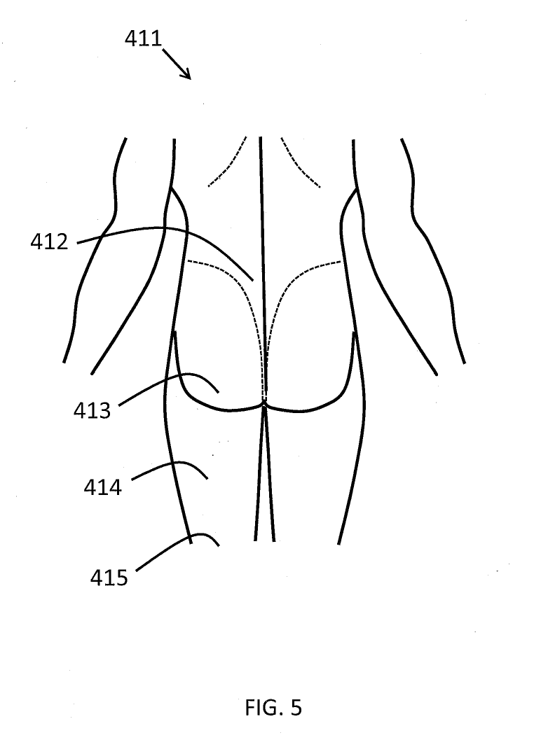

FIG. 4 shows a representation of a human subject 410 seated on a toilet seat, configured as shown in FIGS. 3A & 3B, in a position suitable for light therapy, according to some embodiments of the present invention. The therapeutic light irradiates areas of skin of the human subject exposed within the aperture 308 of the toilet seat when the human subject is seated on the toilet as shown in FIG. 4. The specific areas of skin that receive the light therapy are shown in FIG. 5, which shows a human anatomical dorsal view 411. The targeted portion of skin is located on the human posterior between the lumbus 412, commonly referred to as the lower back and the popliteal fossa 415, commonly referred to as the back of the knee. Specifically the targeted exposed skin is covering the buttocks 413, also known as the gluteal region, and the posterior thigh 414, more commonly referred to as the back of the upper leg.

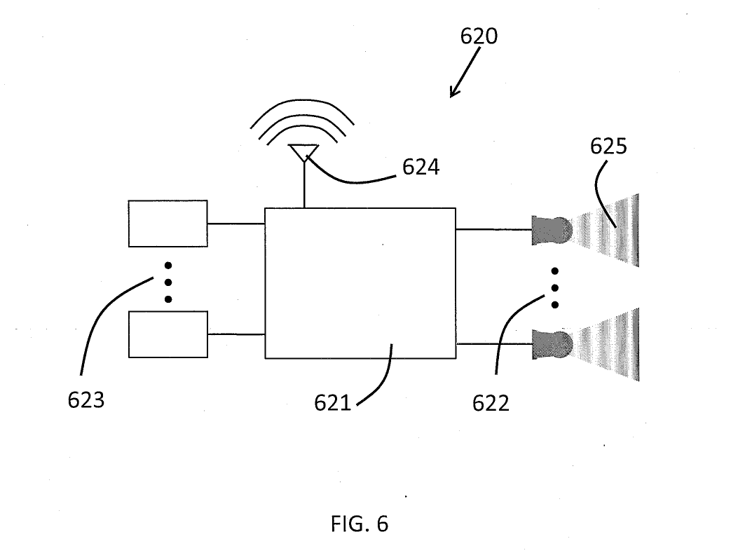

FIG. 6 shows a schematic representation of a control system 620 for a therapeutic light enabled toilet, according to some embodiments of the present invention. The control system comprises a controller 621, at least one sensor 623, optional short range wireless antenna 624, and one or more light emitters 622 emitting therapeutic radiation 625. The sensor(s) and light emitters may be incorporated into the standoffs 106/306 as described above. Data from the sensor(s) such as pressure, skin pigmentation and weight can be fed to the controller to enable/disable one or more light emitters and adjust the exposure duration and/or intensity. The controller may be attached to the bowl (hung outside) or integrated into the bowl (most of a toilet is empty space, so there is plenty of room within) or in embodiments it could be integrated into a compartment in the seat itself or in one of the standoffs. Furthermore, a power supply for the controller/sensors/light emitters may be a battery, or the mains power may be used (particularly when the toilet already has other electrical features, such as a heated seat, etc.).

According to some embodiments, a method of irradiating with therapeutic light a portion of a person's skin located on the posterior between the lumbus and the popliteal fossa during use of a toilet may comprise: providing one or more sensors located on the toilet and detecting the presence of a seated individual; providing one or more light emitters located on the toilet and emitting light toward the seated individual's buttocks and/or upper thigh; providing a controller obtaining information for the sensor(s) and enabling and disabling the light source(s) in response to the sensor data; providing a timer connected to the controller and enabling and disabling by the controller of the light source(s) in response to the timer. Furthermore, providing sensors for detection of skin pigmentation and adjusting, by the controller with access to the pigmentation information, either the duration of the exposure or the intensity of the emitted light to provide a desirable dose.

An example of the use of a therapeutic light enabled toilet according to some embodiments is provided herein and utilizes an embodiment of the toilet with both sensors and light emitters as described above. When no individual is present, a pressure sensor incorporated into the toilet seat, such as described above, informs the controller that it should keep the light emitters 622 disabled, emitting no light. The controller 621 may query the sensor at intervals in a process generally known as polling. In this application a reasonable polling interval would be less than 1 minute. Alternatively, the sensor may be configured to interrupt the controller when the pressure changes by more than a set threshold. This is generally known as interrupt driven sensing and activation. Both polling and interrupt are well known to those with knowledge of sensors and controllers. After the sensor, by polling or interrupt, has indicated to the controller that someone is seated, the controller checks the total pressure reported and quantifies it in a range comprised of at least 8 unique values. More values correspond to a finer resolution of weight applied to the sensor and it is not unusual to have sensors with sensitivities to return the pressure using 8-bit, 12-bit or 16-bit binary values, corresponding to 256, 4096, or 65,536 unique values, respectively. In the present embodiment, a 12-bit sensor is used and the lower 4 bits are ignored providing 256 unique values. Ignoring the lower bits of a pressure sensor provides a method for filtering small variations in pressure due to daily variations in a person's weight, position on the seat, etc., although has sufficient resolution to distinguish between multiple individuals within a household. The controller uses this pressure number to check if this individual has already had an exposure in the last 24 hours and if so, what the cumulative exposure has been. If the exposure limit has not been reached the controller then checks the remaining sensors (if present). The pigmentation sensor is queried to obtain a digital value, with at least 5 levels, corresponding to the presented skin pigmentation. In this embodiment the controller receives 8 levels with the number 0 corresponding to very fair skin and 7 corresponding to extremely dark skin. All other skin pigmentations are at corresponding gradient values between 0 and 7. The controller algorithm can then increase the exposure time and/or intensity linearly with the darkness of the skin to keep the exposure below a target MED limit (Exposure to UV light is generally measured in independent units of a SED, standard erythemal dose. Each individual, based on their exposure history, pigmentation of the skin, age, and other factors has a tolerance measured in a MED. A MED is a minimum erythemal dose, and corresponds to the exposure threshold where skin will react by producing a noticeable pinkening or darkening In general it is desirable to keep to below a 0.5 MED daily exposure to minimize the potential for skin pigmentation changes.) Finally, if a wireless communication interface is present and the corresponding mobile device is detected, user information can be exchanged to get more precise details regarding the desired exposure. Specifically, in combination with the pressure sensor data and skin pigmentation, the mobile device forms a fairly precise identification for tracking daily exposure of an individual. The mobile device may have a desired target exposure, for example 0.7 MED, and a skin pigmentation adjustment or override for the optional pigmentation sensor, for example 0=Fair, 4=Medium, 7=Dark. Finally, the wireless interface can be used to provide the daily total for the person, example a status bar from 0 to 10 with 0 corresponding to no exposure and 10 indicating that the exposure limit has been reached.

For further details of skin pigmentation measurement and corresponding exposure adjustments, and other processes and devices discussed herein, see PCT Publication No. WO 2016/007798 A2 for Wearable Therapeutic Light Source, filed Jul. 9, 2015, incorporated by reference in its entirety herein. Furthermore, this reference also describes the use of a controller to adjust light exposure of the skin based on a person's profile, including but not limited to weight, skin color; and preference regarding the desired exposure level, which may be used in the embodiments of the present invention to target exposure duration and/or intensity for the seated individual. In addition, according to embodiments, the controller may also transmit, or allow a mobile communication device access to, stored information regarding the history of exposures times, durations, and intensities for a specific person. In addition, this reference also describes a controller taking input from sensors to determine exposure duration and intensity. For example, in embodiments a sensor to detect skin pigmentation can be used to adjust reduce the exposure duration and/or intensity for light skin colors and increase the exposure duration and/or intensity for dark skin pigmentation. Yet furthermore, this reference describes a procedure for sensors to be used to calibrate the light emitters in the light source when no individual is seated on the toilet, to adjust the intensity to compensate for manufacturing differences of light sources such as light emitting diodes and the inevitable dimming which occurs with all LED devices, allowing the service life to be extended and more precisely control the dose.

Although specific embodiments of the present invention have been described with light emitters and sensors built into the standoffs attached to the underside of the toilet seat, further embodiments are envisaged in which the light emitters may be positioned under the seat in addition to the standoffs but shorter than the standoffs such that they bear no weight and avoid contact with the main body of the bowl. Furthermore, in embodiments the emitters may be combined with the sensors as a single unit.

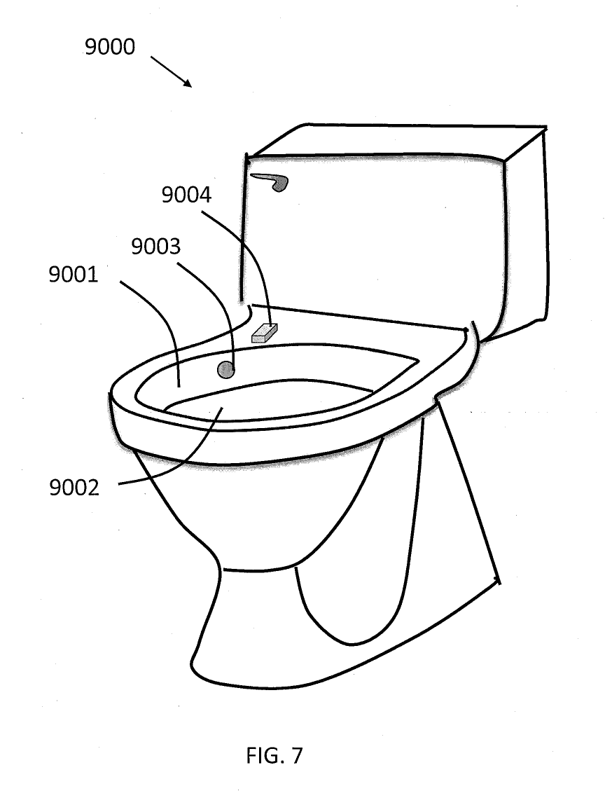

Furthermore, in some embodiments the light emitters/sensors may be positioned on the main body of the bowl, on the rim of the bowl, for example. FIG. 7 shows a toilet 9000 (with seat omitted for clarity) having a bowl rim 9001 which remains dry during flushing and has one or more sensors/emitters (9003) on the vertical surface or the sensor/emitter (9004) as shown on the horizontal surface. Note that surface (9002) is the wet inner surface of the bowl for a flush toilet and further down in the bowl may be found standing water—the sensors/emitters are not attached or integrated into the bowl in these wet areas.

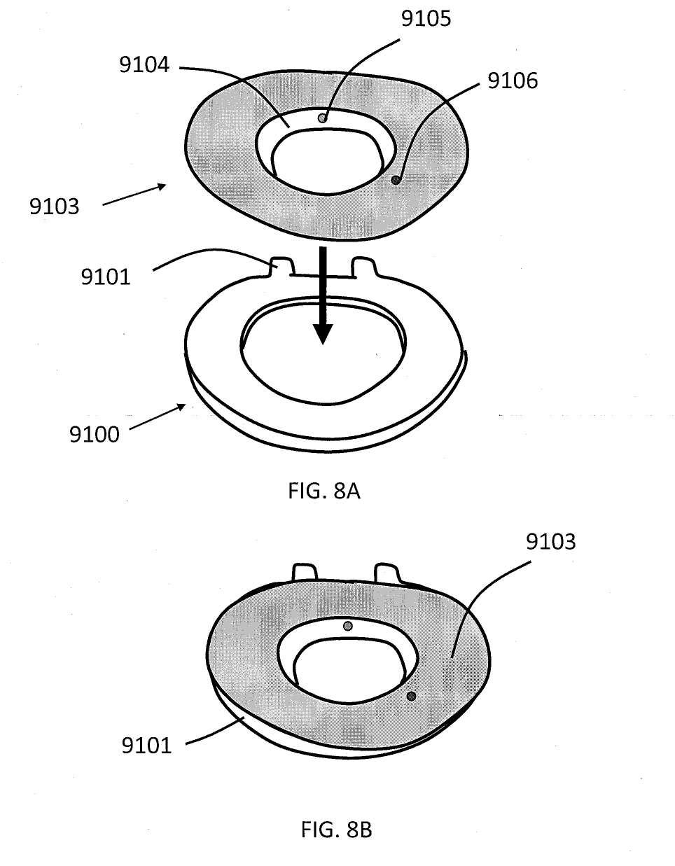

Yet furthermore, in some embodiments the light emitters and/or sensors may be positioned on an insert which is then attached to the toilet seat or the rim of the bowl. The insert may be formed of a high impact plastic material, for example, with wires encapsulated within the insert for connecting sensors/light emitters on the insert to controller and power supply. For example, FIGS. 8A & 8B show a basic typical toilet seat (9100) with hinges (9101) and an insert (9103) that is held in place with a vertical flange (9104) that inserts in the aperture of the seat. Additionally, the figures show possible sensor or emitter locations 9105 and 9106, on or behind the vertical flange and on the surface of the insert, respectively. FIG. 8A shows an exploded arrangement and FIG. 8B shows the insert in position upon the toilet seat.

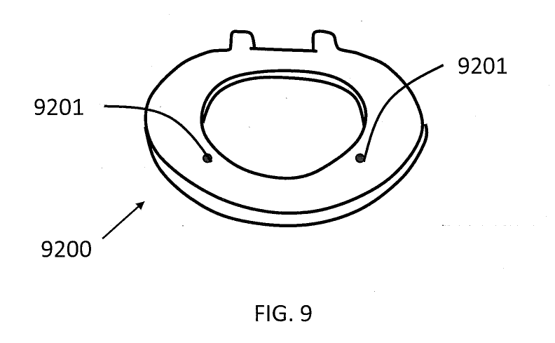

In embodiments, the toilet seat utilizes capacitive sensors to detect contact by a person at one or more positions, these sensors are embedded in the topside of the toilet seat. FIG. 9 shows an example of locations for capacitive sensors (9201) located on a toilet seat (9200). Furthermore, in embodiments the whole toilet seat can be configured as a sensor, but if discrete sensors are used (as shown in FIG. 9) they are preferred to be located in positions most-likely to be in contact with the back of the leg of a person when seated on the toilet seat.

In one embodiment one or more sensors or light emitters may be located on the inside of the main body of the bowl along the upper rim avoiding the portion of the bowl that contains standing water and moving water while flushing.

Although embodiments of the present disclosure have been particularly described with reference to certain embodiments thereof, it should be readily apparent to those of ordinary skill in the art that changes and modifications in the form and details may be made without departing from the spirit and scope of the disclosure.

What is claimed is:

1. A device for therapeutic irradiation with light of a portion of a person's skin located on the posterior between the lumbus and the popliteal fossa during the use of a toilet, comprising:
a light source;
a controller electrically coupled to said light source, said controller being configured to control the intensity of light emitted from said light source and the duration of emission of light from said light source during a therapeutic session;
one or more sensors comprising a first sensor for detecting a person seated on said toilet, said first sensor being electrically coupled to said controller; and
a power source electrically coupled to said light source and said controller;
wherein said controller is further configured to turn on, and keep turned on for said duration of said therapeutic session, said light source when said first sensor detects said person seated on said toilet, and wherein said light source is configured to illuminate said portion of said person's skin located on the posterior between the lumbus and the popliteal fossa.

2. The device of claim 1, wherein at least one of said light source and said one or more sensors are attached to a toilet seat of said toilet.

3. The device of claim 1, wherein at least one of said light source and said one or more sensors are integrated in a toilet seat of said toilet.

4. The device of claim 3, wherein said first sensor is a capacitive sensor and said capacitive sensor is integrated in the top surface of said toilet seat.

5. The device of claim 1, wherein at least one of said light source and said one or more sensors are attached to a bowl of said toilet.

6. The device of claim 1, wherein at least one of said light source and said one or more sensors are integrated in a bowl of said toilet.

7. The device of claim 1, wherein at least one of said light source and said first one or more sensors are attached to an insert, said insert being attached to one of a toilet seat of said toilet or the rim of a bowl of said toilet.

8. The device of claim 1, wherein said first sensor is a pressure sensor and said pressure sensor has the resolution to provide to said controller a signal representing a range of pressures, allowing said controller to distinguish between different persons of different weights and sizes.

9. The device of claim 1, wherein said first sensor is a pressure sensor and said controller is configured to disable said light source when a measured pressure is less than a specified threshold pressure.

10. The device of claim 1, wherein said first sensor is a pressure sensor and said controller is configured to maintain a cumulative log of duration of illumination of said portion of said person's skin for a specific pressure range, said specific pressure range being a proxy for identifying a specific person.

11. The device of claim 10, wherein said cumulative log of exposure is used by said controller to disable further illumination of said portion of said person's skin when a maximum dose of light radiation has been reached.

12. The device of claim 1, wherein said one or more sensors further comprises a skin pigmentation sensor configured to detect a range of pigmentation for said person's skin.

13. The device of claim 12, wherein said controller is further configured to change said duration of the illumination of said portion of said person's skin corresponding to a detected pigmentation of said person's skin.

14. The device of claim 12, wherein said controller is further configured to change the intensity of said light source corresponding to a detected pigmentation of said person's skin.

15. The device of claim 1, further comprising a near field antenna or short range wireless communications system integrated with said controller for passing information between a mobile communications device and said controller.

16. The device of claim 1, wherein said light source comprises a plurality of light emitters, said plurality of light emitters being configured to illuminate the aperture in said toilet seat.

17. The device of claim 1, wherein said light source emits light of wavelength in the range from 290 nm to 400 nm.

18. The device of claim 1, wherein said light source emits light including light of wavelength 298 nm.

19. A method of irradiating with therapeutic light a portion of a person's skin located on the posterior between the lumbus and the popliteal fossa during use of a toilet, comprising:
detecting the presence of a person seated on said toilet using one or more first sensors connected to a controller;
on detecting the presence of a person seated on said toilet, enabling by said controller of a light source for illuminating said portion of said person's skin located on the posterior between the lumbus and the popliteal fossa with therapeutic light.

20. The method of claim 19, further comprising limiting, by said controller, a duration of said illuminating.

21. The method of claim 19, further comprising measuring skin pigmentation of said portion of said person's skin using a second sensor connected to said controller, and adjusting, by said controller taking account of skin pigmentation information for said person, either a duration of said illuminating or the intensity of light emitted by said light source to provide a desirable dose.

22. The method of claim 19, wherein said first sensor is a pressure sensor and said pressure sensor provides to said controller a signal representing a range of pressures, and said controller distinguishes between different persons of different weights and sizes based on said signal.

23. The method of claim 19, wherein said first sensor is a pressure sensor and said controller disables said light source when a measured pressure by said first sensor is less than a specified threshold pressure.

24. The method of claim 19, wherein said first sensor is a pressure sensor and said controller maintains a cumulative log of duration of illumination of said portion of said person's skin for a specific pressure range, said specific pressure range being a proxy for identifying a specific person.

25. The method of claim 24, wherein said controller disables further exposure when said cumulative log of exposure to therapeutic light indicates a maximum dose of therapeutic light has been reached.

27. The method of claim 19, wherein said light source emits light of wavelength in the range from 290 nm to 400 nm.

28. The method of claim 19, wherein said light source emits light including light of wavelength 298 nm.
